(19) United States
(12) Patent Application Publication
(10) Pub. No.: US 2004/0260477 A1
(43) Pub. Date: Dec. 23, 2004

(54) Regulatory classification system

(76) Inventors: Andreas Dittrich, Weil im Schoenbuch (DE); Martin Trostel, Philosophenweg (DE); Petra Kneip, Bondorf (DE); Bernd Anton Bertenbreiter, Bondorf (DE); Craig Richard Cooper, Chiltern Drive Fremont, CA (US); Ray Sato, San Jose, CA (US); Phil Wong, Milpitas, CA (US); Ed Lungren, Sunnyvale, CA (US); Hiddo Gerhard Boelkes, Ehningen (DE); Gregory R Felton, Mill Valley, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/490,199

(22) PCT Filed: Oct. 1, 2001
(86) PCT No.: PCT/US01/30702

(51) Int. Cl. G06F 19/00; G01N 33/48; G01N 33/50; G06F 7/00; G06F 17/00

(52) U.S. Cl. 702/19; 707/100

(57) ABSTRACT

according to one aspect of the present invention, regulatory classification of a product is performed by using a computer to search a database for at least one classification covering the product; outputting a classification if the classification is found in the database; and, if no classification is found, ascertaining at least one classification in a different manner and entering any ascertained classifications to the database.

### BACKGROUND OF THE INVENTION

Many countries require product classification for international trade. Product classification is used for duty and tax calculations, import and export licensing, hazardous materials documentation, trade statistics, and a variety of other purposes.

Large companies usually maintain staffs of administrators who perform product classification. Before a product can be exported or imported, an administrator must classify the product.

Classifying the product can be slow and manually intensive, especially for a company that sells a large number and variety of products to many different countries. Not only does the classification involve importing exporting country laws, but it usually involves importing country laws as well.

Much of the classification work is redundant, as different experts often classify the same or similar products and parts. Moreover, the experts might use different tools and standards. Inconsistent classifications can result.

### SUMMARY OF THE INVENTION

According to one aspect of the present invention, regulatory classification of a product is performed by using a computer to search a database for at least one classification covering the product; outputting a classification if the classification is found in the database; and, if no classification is found, ascertaining at least one classification in a different manner and entering any ascertained classifications to the database.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is an illustration of a method of performing regulatory classification of a product.

FIG. 2 is an illustration of an exemplary record for a classification database.

FIG. 3 is an illustration of an entry in a conversion table.

FIG. 4 is an illustration of a method of performing an audit check.

FIG. 5 is an illustration of a classification system.

### DETAILED DESCRIPTION OF THE PREFERRED EMBODIMENTS

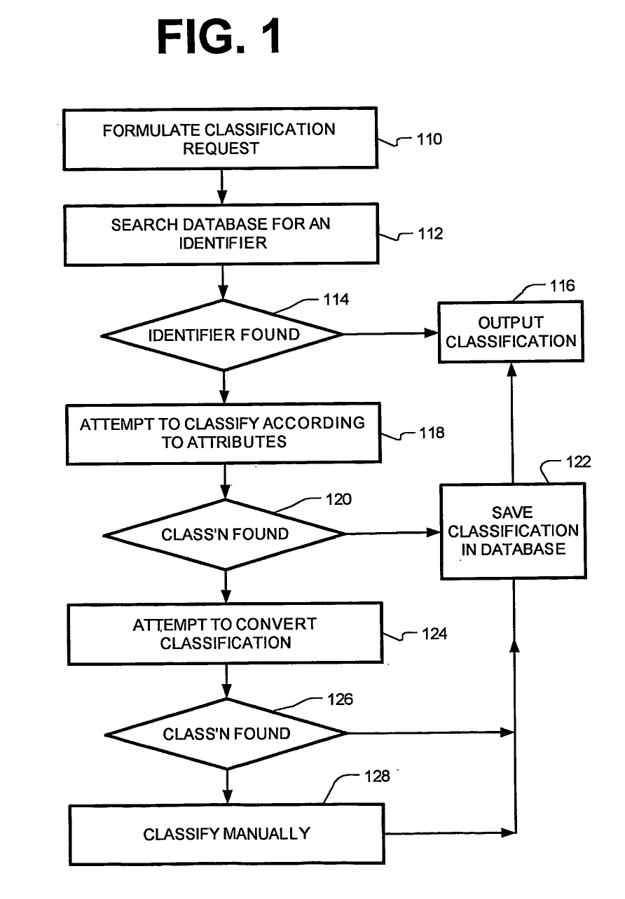

Reference is made to FIG. 1, which illustrates a method of performing regulatory classification of a product. First, a classification request is formulated (block 110). Included in the classification request is information about the product. The product information might include a product name, a product number or other identifier.

The classification request may also specify search criteria, such as region, subject matter group, and the reason for classification. For example, a classification request could request classification of a machine bolt for import into the United States with respect to a specific subject matter group. U.S. subject matter groups include import classification (e.g., Harmonized Tariff Classification), export classification (e.g., Export Commodity Control Number), toxic goods (e.g., Toxic Goods Control Act Classification), etc. Regions could include geographic regions (e.g., European Union) and specific countries (e.g., Germany). Regions could also include standards bodies (e.g., standards for electrical security, FCC), which are not geographically bound.

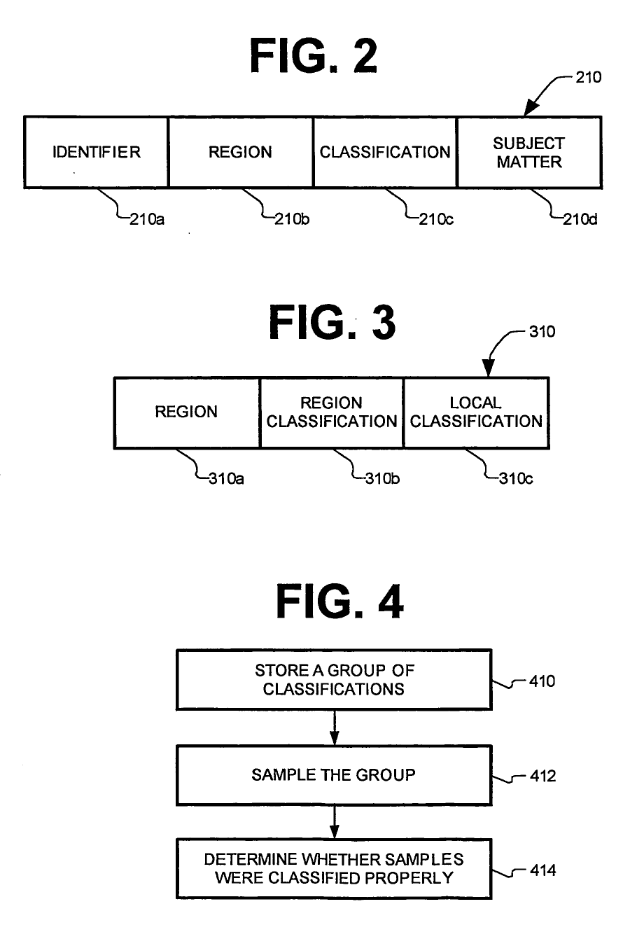

A computer is used to search a database for at least one classification covering the product information (block 112). Each record 210 in the database may include an identifier 210a, a classification 210b, a region 210c, and a subject matter group 210d (see FIG. 2). Classifications for different subject matter groups may be searched if a subject matter group is not specified in the classification request. Thus, different subject matter classifications can be found for a single product or component.

Classifications for different regions may be searched if a region is not specified in the classification request. Typically, classifications for the following two countries will be searched: the importing country, and the exporting country. Different countries usually have different classification groups. Thus, different country classifications can be found for a single product or component.

A classification is found (block 114) if there is an exact match between the identifier in the classification request and an identifier in a database record (and all other search criteria is satisfied). A classification could be found if the identifier in the classification request is similar to the identifier in the database record, provided that products having similar identifiers are assured of being in the same class. Such an assurance may be provided, for example, by a strict product numbering policy.

If an identifier is found (block 114), the corresponding classification or classifications are outputted (block 116). The classifications may be outputted by outputting a file with the classification, displaying (e.g., printing, showing on a video monitor) the classification, providing the classification back to a linked application, etc. The identifier or other product information may be outputted with the classifications. Outputting the classifications may include preparing import declarations, export declarations, toxic substance declarations, and other declarations according to the subject matters supported by the classification system.

If the identifier is not matched, the classification may be ascertained in a different manner. A classification may be derived from attributes of the product (block 118). Exemplary attributes include, but are not limited to, size, length, weight, capacity, resistance, performance data, whether the product includes software, whether it is recorded on CD, tape, or disc, etc. A decision engine for this attribute-driven classification may be built as a decision tree, where the answer to a question determines the next set of questions to be asked, or the classification if enough details have been gathered. Exemplary questions for a screw are as follows.

1) Type of screw (Self-tapping, Machine, Stud, other):

2) ‘Made of’ material (of both shank and head): If shank is steel, is it stainless steel?

3) Diameter of shanks or threads in mm:

4) Length of screw in mm (not including cap):

5) Type of head (round, hexagonal, etc.):

These questions may be prepared by a classification expert. If answers to the questions allow a classification to be found, (block 120), the corresponding classification is saved in the database (block 122) and outputted (block 116).

If the attempt at the attribute-driven classification fails, an attempt may be made to convert the classification from one region to another. If a classification is found in one region but not another (e.g., the exporting country but not the importing country), the classification may be derived from the classification of the same product or component in a different region (block 124). A lookup table may be used to convert the classification of one region to the classification of another region. An entry 310 in the lookup table is shown in FIG. 3. The entry 30 identifies the local classification 310a, a different region 310b, and a corresponding classification of that different region 310c Any converted classifications are entered in the database (block 122) and outputted (block 116).

If, for a product or component, all classifications for all requested subject matter groups for all relevant regions still have not been found (block 126), the classification is placed on hold for review and approval by an expert (block 128). When a classification request is placed on hold, the classification request is routed to the appropriate expert, depending upon area of expertise, geographic coverage, product and technical knowledge, and priority of request. Classifications found by manual review are stored in the database (block 122).

If the search request does not specify a subject matter group, then all subject matter groups for a specified region will be searched. A lookup table may be used to identify all of the subject matter groups for each region.

The method of FIG. 1 is performed on each product indicated in a classification request. If components of the product are classified, a separate classification request may be provided for each component. In that case, the product information would describe a component, and the method of FIG. 1 would be performed on each component.

The converted and attribute-driven classifications may be routed to a classification expert for approval, before they are finally saved and outputted. That way, the converted and attribute-driven classifications would be ‘proposals’ requiring expert approval (or modification).

The attribute-driven classification and the classification conversion are optional. If both of these functions are performed, the order in which they are performed may be different than the order shown in FIG. 1. For instance, the classification conversion may be performed before the attribute-driven classification.

Eventually, enough records will be added to the database whereby most classifications will be performed automatically, without manual support. Thus, as the database grows larger, the speed of obtaining classifications will be increased and the need for manual support will be reduced. Moreover, repeatedly classifying the same or similar products/components is eliminated. The savings in time and manpower is especially advantageous for a company that deals with large quantities of products each day, with each product having many different components.

If the database is accessible 24 hours a day, classifications can be obtained at any time. If the database is made available via a server having a standard interface (e.g., a web-based interface), the classifications can be obtained conveniently.

The database may be used in ways other than obtaining classifications. Audits, quality checks, and classification comparisons may be performed. Classification consistency may be measured, for example, by comparing the classifications from different countries, or different regulations. Business models may be systematically analyzed and evaluated for potential duty and tax savings.

The computer may be programmed to perform automated duty recovery. The process for calculating the duty recovery could be linked with the classification system, and from there retrieve historical classifications and/or duty rates and calculate the differences to be paid or requested.

FIG. 4 illustrates a method of performing an audit check. A group of the classification results is stored in a database (block 410), the group is randomly sampled (block 412), and a determination is made as to whether the sampled classification results were determined properly (block 414).

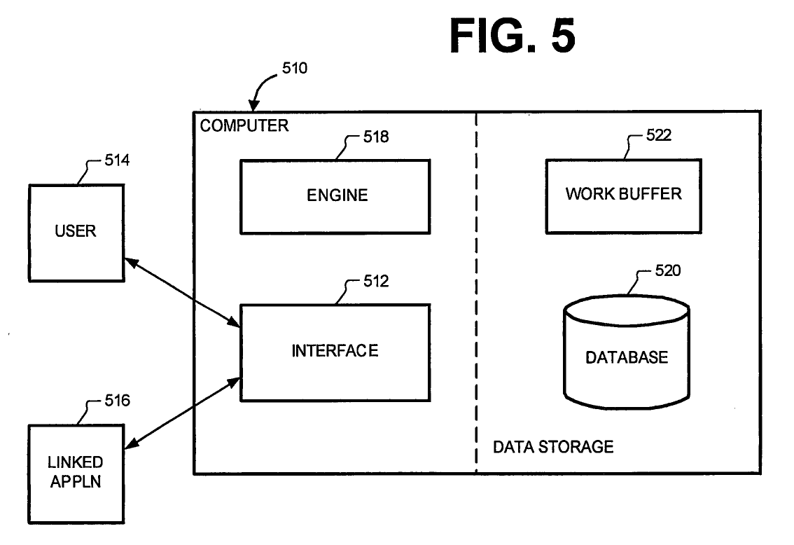

Reference is made to FIG. 5, which shows a system including a computer 510 programmed to perform the regulatory classification. The computer 510 provides an interface 512 for receiving classification requests from sources such as users 514 and linked applications 516.

The interface 512 allows classification experts to request, assign, review and approve classifications, as well as enter questions for the attribute-driven classification. The interface 512 allows users supporting or executing different business processes (e.g., sales reps, order administration, logistics personnel) to look up classifications and submit requests for classifications. It allows business partners such as customers and suppliers to look up classifications and request classifications. The interface 512 allows classifications to be assigned to multiple products as a single user activity, as opposed to assign product-by-product.

The computer 510 also provides an engine 518. The engine 518 performs the functions shown in FIG. 1.

A database 520 stores classification records, and it may also maintain product and classification documentation. The database 520 also may store the questions that are used for attribute-driven classification. The information stored in the database 520 may be used to determine whether reasonable care has been applied when assigning classifications.

The database 520 may store, maintain and provide information on classification schedules and tariffs, and duty tax rates. This allows the computer 510 to systematically analyze and evaluate business models for potential duty and tax savings.

Classification requests for classifications that need manual support may be placed in a work buffer 522 and remain in the work buffer 522 until classification has been completed or cancelled. The classification requests in the work buffer 522 may be grouped and sorted organized according to requesting organization and function; requesting individual; requesting date; assigned priority; product attributes such as descriptions and product categories, requested classification subject area; or requested region. Such an organization makes it easier for an individual to find an entry in the work buffer.

The database 520 and the work buffer 522 may be stored in the computer, as shown in FIG. 5. In the alternative, the database 520 and the work buffer 522 may be stored in a standalone device (e.g., a network attached storage device) or another machine (e.g., another computer).

The database 520 may be a central database. Use of a central database offers a single repository for product classification. The central database ensures that the same products receive the same classifications.

The present invention is not limited to a single computer for performing the regulatory classification. Instead, the regulatory classification may be run on a distributed logic/data system having several computers.

The regulatory classification may be performed by a dedicated system, or it may be integrated into another system, such as an order management system. For example, the order management system might need export control numbers to determine whether an export license is needed for a particular transaction. The order management system might also make use of classifications for quotation, sales and marketing, order processing, procurement, importation, duty drawback, shipping, and hazardous materials documentation.

Although a specific embodiment of the present invention has been described and illustrated, the present invention is not limited to the specific forms or arrangements of parts so described and illustrated. Instead, the present invention is construed according to the claims that follow.

What is claimed is:

1. A method of performing regulatory classification of a product, the method comprising:
using a computer to search a database for at least one classification covering the product; if a classification is found in the database, outputting the classification;
if no classification is found, ascertaining at least one classification in a different manner and entering any ascertained classifications to the database.

2. The method of claim 1, wherein the different manner includes performing a manual determination of the classification.

3. The method of claim 2, wherein product information for performing the manual determination is placed in a work buffer.

4. The method of claim 1, wherein the different manner includes deriving the classification from attributes of the product.

5. The method of claim 5, wherein classification is derived by using the computer to prompt a user for questions about the attributes of the product, and using the computer to attempt to classify the product based on answers to the questions.

6. The method of claim 1, wherein the different manner includes deriving the classification from the classification of the same product of another region.

7. The method of claim 1, wherein the computer searches for different subject matter groups.

8. The method of claim 1, wherein the computer searches for different regions.

9. The method of claim 1, further comprising storing a plurality of classification results; randomly sampling a group of the classification results, and determining whether the sampled classification results were determined properly.

10. A regulatory classification system comprising:
an interface for receiving classification requests, the classification requests including product information;
a database; and
an engine for searching the database for at least one classification covering the product information, the engine outputting a classification if the classification is found in the database; and, if no classification is found, the engine allowing the classification to be ascertained in a different manner, any ascertained classifications being added to the database.

11. The system of claim 10, further comprising a work buffer for storing classification requests needing manual attention.

12. The system of claim 10, wherein the different manner includes performing an attribute-driven classification.

13. The system of claim 12, wherein the engine prompts a user for questions about the attributes relating to the product information, and attempts to classify the product based on answers to the questions.

14. The system of claim 10, wherein the different manner includes deriving the classification from classification of the same product of another region.

15. The system of claim 14, further comprising a lookup table for converting the classification of one region into the classification of another region.

16. The system of claim 10, wherein the engine searches the database for different subject matter groups.

17. The system of claim 10, wherein the engine searches the database for different regions.

18. The system of claim 10, wherein the engine searches the database for product identifiers.

19. The system of claim 10, wherein the engine stores classification results; randomly samples a group of the classification results, and determines whether the sampled classification results were determined properly.

20. Apparatus for using a database to perform regulatory classification, the classification being performed in response to a classification request, the request including product information, the apparatus comprising an engine for searching the database for at least one classification covering the product information, the engine outputting a classification if the classification is found in the database; and, if no classification is found, the engine allowing the classification to be ascertained in a different manner, any ascertained classifications being added to the database.
